(19) United States
(12) Patent Application Publication
(10) Pub. No.: US 2007/0135773 A1
(43) Pub. Date: Jun. 14, 2007

(54) Wash water storage apparatus for portable irrigator

(76) Inventor: Byung Eun Yoo, Daegu (KR)

Correspondence Address: CANTOR COLBURN, LLP, 55 GRIFFIN ROAD SOUTH, BLOOMFIELD, CT, 06002, US

(21) Appl. No.: 11/439,841

(22) Filed: May 24, 2006

(30) Foreign Application Priority Data
Dec. 8, 2005 (KR) 20-2005-0034573

(51) Int. Cl. A61M 31/00

(52) U.S. Cl. 604/275; 604/279

(57) ABSTRACT

Disclosed herein is a wash water storage apparatus for use with a portable irrigator which can remove impurities contained in wash water to be supplied into the irrigator and also can keep the wash water at a constant temperature, thereby supplying harmless wash water to a human body. The wash water storage apparatus for supplying wash liquid such as wash water into the irrigator that is usable to wash the interior of the vagina or intestine, comprises an outer body having a cover provided at an upper end thereof and configured to be pivotally opened from and closed to the outer body by means of a hinge shaft, and grip recesses, a power switch, temperature regulation switches, and a temperature gauge board which are provided at a middle region thereof, a filtering bucket configured to be inserted into and received in the outer body, and having a filter, and a supporting base mounted at a lower end of the outer body and having a heater installed to come into contact with a lower surface of the filter and adapted to heat the wash water supplied from the filtering bucket into the filter, at least one water level sensor and water temperature sensor for detecting the level and temperature of the wash water, and a wash liquid drain channel to be connected to a wash liquid supply hose of the irrigator.

### BACKGROUND OF THE INVENTION

1. Field of the Invention

The present invention relates to a wash water storage apparatus for portable irrigator that serves to remove feces contained for a long time in the human large intestine or wash the interior of the women's vagina, and more particularly, to a wash water storage apparatus for portable irrigator, which can remove impurities contained in wash water to be supplied into the irrigator and also can keep the wash water at a constant temperature, thereby supplying harmless wash water to a human body.

2. Description of the Related Art

As well known, the human large intestine has functions of not only regulating the content of moisture and maintaining a predetermined amount of electrolyte, but also making feces from ingesta by bacterial action. However, if the large intestine is not functioning properly, harmful ingredients and poisonous substances are accumulated in the human body, and thus, intestinal paralysis as well as expansion or rupture of aged blood vessels may be caused. As a result, the large intestine may be a hotbed of bacteria and various intestinal diseases such as constipation, diarrhea, indigestion, slim feces, abdominal intumescence, large intestine cancer, colitis, anaphylaxis syndrome, ulcer of the large intestine, piles, etc.

Accordingly, since accumulation of waste materials in the intestines causes intestinal paralysis, malfunction of the intestines, skin aging, and other various diseases, there is the urgent need for appropriate measures to eliminate the causes of the various intestinal diseases. For this reason, conventional intestinal irrigators have been used to remove the waste materials in the intestines and consequently prevent the various intestinal diseases. The conventional intestinal irrigators have been found to obtain outstanding effects in the removal of poisonous substances caused by feces contained for a long time in the large intestine, and therefore, are being widely used for the prevention and treatment of various intestinal diseases.

As an example of the conventional intestinal irrigators, Korean Utility Model Registration Nos. 338,146 and 342,570, which are registered by the applicant of the present invention, disclose portable irrigators having advantages of simple and convenient use with no cumbersome use feeling. However, the disclosed portable irrigators have problems in that a water container, in which wash water to be supplied into the irrigator is received, has a poor outer appearance and also is undesirable in view of sanitation because it has no function of preventing invasion of impurities into the received wash water.

Furthermore, the disclosed container has a problem of inconvenience in use.

### SUMMARY OF THE INVENTION

Therefore, the present invention has been made in view of the above problems, and it is an object of the present invention to provide a wash water storage apparatus for exclusive use with a portable irrigator used by a patient or woman for the washing of the large intestine or vagina, which can remove impurities contained in wash water to be supplied into the irrigator and also can appropriately regulate the temperature of the wash water.

In accordance with the present invention, the above and other objects can be accomplished by the provision of a wash water storage apparatus for supplying wash liquid such as wash water into a irrigator that is usable to wash the interior of the vagina or intestine, comprising: a cover provided at an upper end of the outer body and configured to be pivotally opened from and closed to the outer body by means of a hinge shaft, and an outer body composed of grip recesses, a power switch, temperature regulation switches, and a temperature gauge board which are provided at a middle region thereof, a filtering bucket configured to be inserted into and received in the outer body, and having a filter; and a supporting base mounted at a lower end of the outer body and having a heater installed to come into contact with a lower surface of the filter and adapted to heat the wash water supplied from the filtering bucket into the filter, at least one water level sensor and water temperature sensor for detecting the level and temperature of the wash water, and a wash liquid drain channel to be connected to a wash liquid supply hose of the irrigator.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a side elevation view illustrating a state wherein a filtering bucket is lifted upward through an opened cover of a wash water storage apparatus according to the present invention;

FIG. 2 is a longitudinal section view taken along the line I-I of FIG. 1;

FIG. 3 is a top plan view of FIG. 1 having no filtering bucket;

FIG. 4 is a top plan view illustrating the wash water storage apparatus according to the present invention, which is connected to a portable irrigator;

FIG. 5 is a cross sectional view taken along the line II-II of FIG. 4;

FIG. 6 is an enlarged sectional view illustrating a irrigator that is connectable to the wash water storage apparatus according to the present invention; and

FIGS. 7A and 7B are views illustrating different embodiments of a nozzle injector included in the irrigator.

### DESCRIPTION OF THE PREFERRED EMBODIMENTS

Now, preferred embodiments of the present invention will be explained in detail with reference to the accompanying drawings.

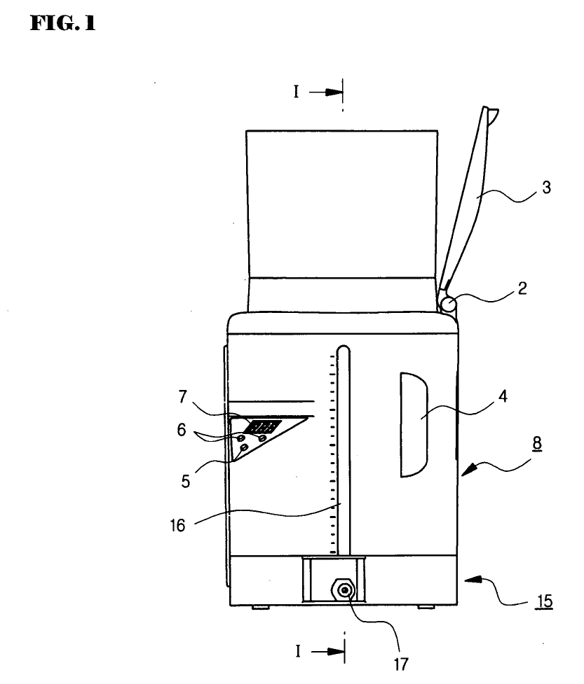

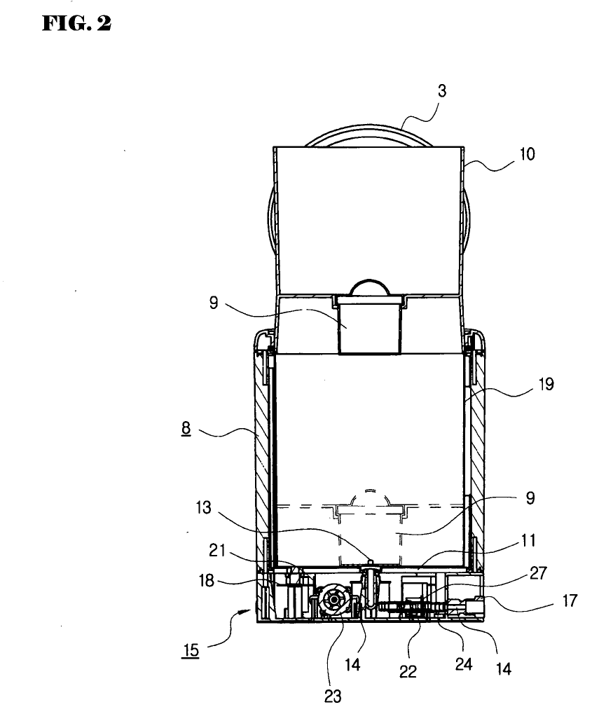

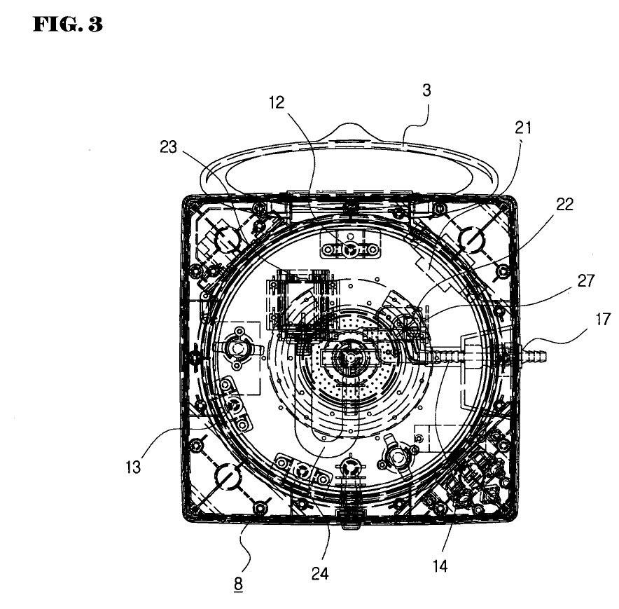

FIGS. 1 and 2 are a side elevation view and a longitudinal section view, respectively, illustrating a state wherein a filtering bucket is lifted upward through an opened cover of a wash water storage apparatus according to the present invention, and FIG. 3 is a top plan view illustrating a state wherein the filtering bucket is removed from the wash water storage apparatus.

The wash water storage apparatus of the present invention, which is designated as reference numeral 1, is used to supply wash liquid such as wash water into a irrigator 100 that is usable to wash the interior of the vagina or large intestine. The wash water storage apparatus 1 basically includes an outer body 8, a filtering bucket 10 configured to be inserted into and received in the outer body 8, and a supporting base 15 mounted at a lower end of the outer body 8. More particularly, the outer body 8 is provided at an upper end thereof with a cover 3 configured to be pivotally opened from and closed to the outer body 8 by means of a hinge shaft 2. The outer body 8 is also provided at a middle region thereof with grip recesses 4, a power switch 5, temperature regulation switches 6, and a temperature gauge board 7. The filtering bucket 10 is internally provided with a filter 9. The supporting base 15 includes a heater 11 installed to come into contact with a lower surface of the filter 9 and adapted to heat the wash water supplied from the filtering bucket 10 into the filter 9. The supporting base 15 also includes at least one water level sensor 12 and water temperature sensor 13 for detecting the level and temperature of the wash water, and a wash liquid drain channel 14 to be connected to a wash liquid supply hose 101 of the irrigator 100.

The cover 3 of the outer body 8, which is pivotally opened and closed by means of the hinge shaft 2, is made of a transparent material. Also, the outer body 8 is provided with a water level window 16 for visually inspecting the amount of water received in the filtering bucket 10. Here, the filtering bucket 10 is detachably received in the outer body 8. A one-touch coupling 17 is coupled to an end of the drain channel 14 mounted in the supporting base 15 for the connection of the wash liquid supply hose 101 of the irrigator 100 which will be explained hereinafter.

The heater 11, which is mounted in the supporting base 15, is connected to an overheating prevention bimetal 18. In the wash water storage apparatus of the present invention, a silver-plated body water container 19 is integrally mounted in the outer body 8 for sterilizing harmful bacteria contained in the wash water having passed through the filter 9 of the filtering bucket 10.

With this configuration, if the wash liquid is supplied into the irrigator 100, the wash liquid is primarily filtered by the filter 9, and then, is sterilized by the body water container 19 plating with a silver purity of 99.9% to sterilize harmful bacteria contained in the filtered wash water. Thereby, the wash water storage apparatus of the present invention is able to supply the sterilized clean wash water to a human body, while keeping the wash water at a temperature most suitable for a human body (approximately 38° C.).

The temperature of the wash water can be regulated by using the left and right temperature regulation switches 6 and the temperature gauge board 7. Also, the amount of the wash water can be checked by using the water level sensor 12 and water level window 16. If no wash water remains, the water level sensor 12 detects the exhaustion of the wash water, and commands the heater 11 to be automatically turned off.

The supporting base 15 equipped with the heater 11 is mounted at the lower end of the outer body 8, and is made of an incombustible material.

Meanwhile, the irrigator 100, which is connectable to the wash water storage apparatus 1 of the present invention by use of the wash liquid supply hose 101, includes a wash liquid supply body 106 having an inner wash liquid inflow passage 109 and wash liquid outflow passage 110, which communicate with each other. The wash liquid supply body 106 is formed with a flange 107 for the integral connection of the wash liquid supply hose 101. The irrigator 100 further includes a nozzle injector 103 connected to an end of another wash liquid supply hose 101 that is connected at the other end thereof to the wash liquid supply body 106, and a manual air pump 102 attached to a lower end of the wash liquid supply body 106 below the wash liquid outflow passage 110.

Here, the manual air pump 102 takes the form of a handle portion of a shut gun.

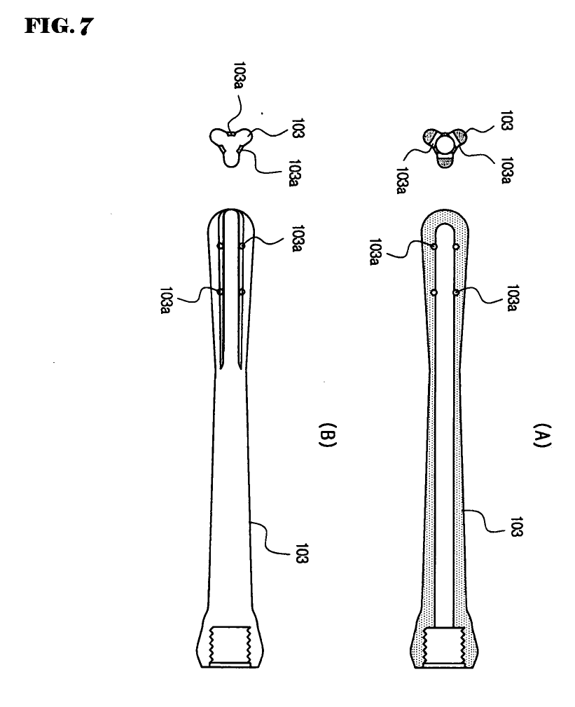

The nozzle injector 103 is a member to be inserted into the anus or vagina. Accordingly, it is desirable that the nozzle injector 103 be detachably connected to the wash liquid supply hose 101 in a screw fastening manner so as to be easily exchangeable when it is polluted. As shown in FIGS. 7A and 7B, a plurality of wash liquid supply holes 103a are formed at a tip end portion of the nozzle injector 103, to achieve high injection efficiency of wash liquid.

The wash liquid supply body 106 is also internally provided with first and second check valves 111 and 112 to ensure the supply of wash liquid in a direction indicated by arrows, without the risk of backflow.

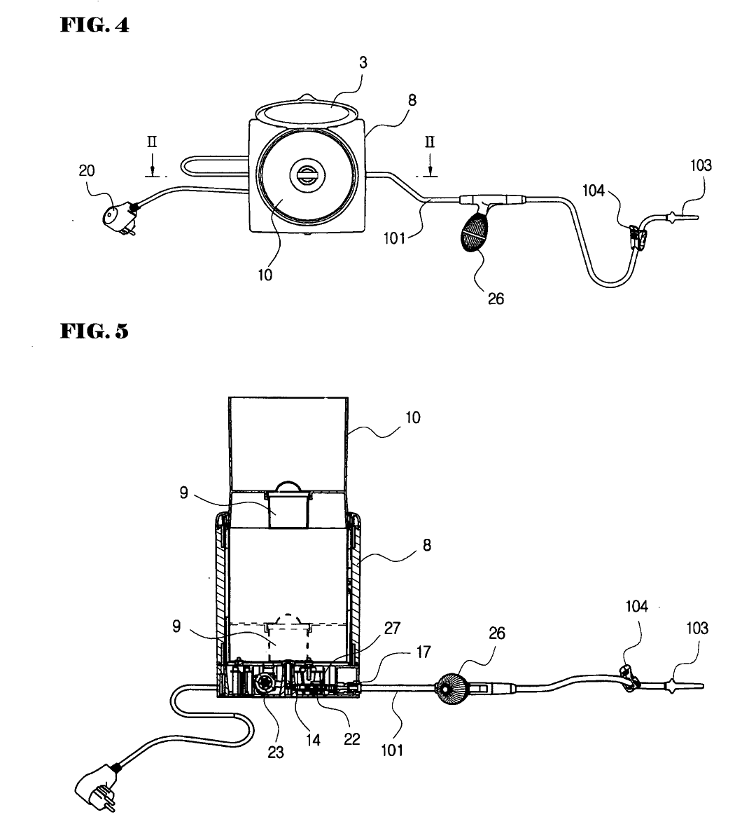

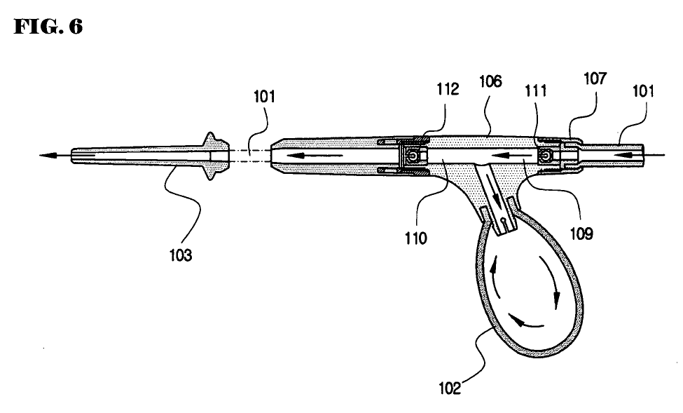

Hereinafter, an embodiment of the present invention will be explained with reference to FIGS. 4 and 5.

In use, first, the wash liquid supply hose 101 of the irrigator 100 is connected to the drain channel 14 of the supporting base 15 that is mounted at the lower end of the outer body 8. In this case, the wash liquid supply hose 101 is smoothly connectable to the drain channel 14 by use of the one-touch coupling 17.

Subsequently, after a power cord 20 is connected to an external power source, the temperature of wash water, which is supplied into the silver-plated body water container 19 by passing through the filtering bucket 10, is regulated to an appropriate value. Thereafter, the wash liquid can be supplied into the intestine or vagina by way of the nozzle injector 3 by regulating the manual air pump 102 of the irrigator 100.

Herein, reference numeral 104 (not explained) indicates a flow rate adjustor.

As apparent from the above description, the present invention provides a wash water storage apparatus for portable irrigator, which is capable of not only filtering and sterilizing wash water to be supplied into the irrigator, but also keeping the wash water at an appropriate temperature, thereby enabling the washing of the interior of the intestine or vagina using the clean and temperature regulated wash water. Further, the wash water storage apparatus of the present invention can achieve good outer appearance and convenience in use, and is easy to store and carry wash water due to a compact size thereof. Accordingly, the wash water storage apparatus of the present invention is advantageously usable together with a portable irrigator.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A wash water storage apparatus for supplying wash liquid such as wash water into a irrigator that is usable to wash the interior of the vagina or intestine, comprising:
an outer body having a cover provided at an upper end thereof and configured to be pivotally opened from and closed to the outer body by means of a hinge shaft, and grip recesses, a power switch, temperature regulation switches, and a temperature gauge board which are provided at a middle region thereof;
a filtering bucket configured to be inserted into and received in the outer body, and having a filter; and
a supporting base mounted at a lower end of the outer body and having a heater installed to come into contact with a lower surface of the filter and adapted to heat the wash water supplied from the filtering bucket into the filter, at least one water level sensor and water temperature sensor for detecting the level and temperature of the wash water, and a wash liquid drain channel to be connected to a wash liquid supply hose of the irrigator.

2. The apparatus as set forth in claim 1, wherein:
the outer body further has a water level window for visually inspecting the amount of water received in the filtering bucket, the filtering bucket being detachably received in the outer body; and
the drain channel, which is mounted in the supporting base, is provided at an end thereof with a one-touch coupling for the connection of the wash liquid supply hose of the irrigator.

3. The apparatus as set forth in claim 1, wherein the heater, which is mounted in the supporting base, is connected to an overheating prevention bimetal.

4. The apparatus as set forth in claim 1, wherein the outer body is internally mounted with a silver-plated body water container for sterilizing harmful bacteria contained in the wash water having passed through the filter of the filtering bucket.

5. The apparatus as set forth in claim 1, wherein the irrigator, which is connectable to the wash water storage apparatus by use of the wash liquid supply hose, includes:
a wash liquid supply body having an inner wash liquid inflow passage and wash liquid outflow passage, which communicate with each other, the wash liquid supply body being formed with a flange for the integral connection of the wash liquid supply hose;
a nozzle injector connected to an end of another wash liquid supply hose that is connected at the other end thereof to the wash liquid supply body; and
a manual air pump attached to a lower end of the wash liquid supply body below the wash liquid outflow passage.

6. The apparatus as set forth in claim 5, wherein a plurality of wash liquid supply holes are formed at a tip end portion of the nozzle injector.
